(19) United States
(12) Patent Application Publication
(10) Pub. No.: US 2021/0290153 A1
(43) Pub. Date: Sep. 23, 2021

(54) DRUG DELIVERY DEVICES HAVING ADSORBED MEDICATION ADHERENCE MARKERS THEREON, PACKAGING INCLUDING THE SAME AND METHODS OF FORMING THE SAME

(71) Applicant: Xhale, Inc., Gainesville, FL (US)

(72) Inventor: Hank Wohltjen, Bowling Green, KY (US)

(21) Appl. No.: 17/261,127

(22) PCT Filed: Jul. 19, 2019
(86) PCT No.: PCT/US2019/042514

§ 371 Date: Jan. 18, 2021

Related U.S. Application Data
(60) Provisional application No. 62/700,771, filed on Jul. 19, 2018.

(51) Int. Cl. A61B 5/00; A61K 9/28; A61K 9/48; A61B 5/08

(52) U.S. Cl. CPC A61B 5/4833; A61K 9/286; A61K 9/4891; A61B 2560/04; A61B 5/082; A61B 5/42; A61K 9/4816

(57) ABSTRACT

Provided herein are pharmaceutical packages that include a pharmaceutical tablet or capsule, and a volatile medication adherence marker (MAM) contained on or in a MAM reservoir external to the pharmaceutical tablet or capsule. Also provided herein are methods of surface coating a pharmaceutical tablet or capsule with a volatile medication adherence marker (MAM) that include placing the pharmaceutical tablet or capsule in a sealed container with the volatile MAM contained on or in a MAM reservoir external to the pharmaceutical tablet or capsule, and maintaining the seal of the container for a sufficient time to allow MAM vapors from the volatile MAM reservoir to adsorb to onto a surface of the pharmaceutical tablet or capsule. Also provided are methods of medication adherence monitoring using such pharmaceutical tablets or capsules.

### CROSS-REFERENCE TO RELATED APPLICATIONS

This application claims the benefit of U.S. Provisional Application No. 62/700,771, filed Jul. 19, 2018, the contents of which are incorporated herein by reference in their entirety.

### FIELD OF THE INVENTION

The present invention relates to pharmaceutical capsules and tablets. The present invention also relates to medication adherence monitoring products and methods.

### BACKGROUND OF THE INVENTION

Non-compliance of patients to drug regimens prescribed by their physicians results in excessive healthcare costs estimated to be around $100 billion per year through lost work days, increased cost of medical care, higher complication rates, as well as drug wastage. In addition, non-compliance of drug regimens by patients during clinical trials may result in denial of FDA clearance for otherwise viable drugs. It is estimated that the average non-adherence rates among patients in clinical trials receiving treatment for chronic conditions can be as high as 57%. See, The New England Journal of Medicine in 2005 (Lars Osterberg, M. D. and Terrence Blaschke, M. D., “Drug Therapy: Adherence to Medication”). If a drug fails to achieve approval in part because participants are not taking the study medication, significant money is wasted and drugs that may be useful to patients never gain approval. Non-compliance refers to the failure to take the prescribed dosage at the prescribed time which results in under-medication or over-medication.

Devices, systems and methods for breath-based monitoring medication adherence are known in the art. Examples of such devices, systems and methods can be found, for example, in U.S. Pat. No. 7,820,108, and U.S. Publication Nos. 2014/0294675, 2010/0255598 and 2014/0341983, the contents of which are incorporated herein by reference in their entirety. Despite the success of such methods, there remains a need in the art for improved products, devices, systems and methods for medication adherence monitoring.

### BRIEF DESCRIPTION OF THE DRAWINGS

FIG. 1 is an illustration of a standard pharmaceutical blister pack.

FIG. 1 is an illustration of a blister cavity of the blister pack shown in FIG. 1.

FIG. 2 shows a simple pharmaceutical package according to an embodiment of the invention. The vial shown includes a tablet coated with maltodextrin and a sorbent pad loaded with ca. 200 mg of 2-butanol.

### DETAILED DESCRIPTION OF EMBODIMENTS OF THE INVENTION

The present invention now will be described more fully hereinafter with reference to the accompanying drawings, in which embodiments of the invention are shown. However, this invention should not be construed as limited to the embodiments set forth herein. Rather, these embodiments are provided so that this disclosure will be thorough and complete, and will fully convey the scope of the invention to those skilled in the art.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

It will be understood that when an element is referred to as being “on” or “adjacent” to another element, it can be directly on or directly adjacent to the other element or intervening elements may also be present. In contrast, when an element is referred to as being “directly on” or “directly adjacent” to another element, there are no intervening elements present. It will also be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Like numbers refer to like elements throughout the specification.

It will be understood that, although the terms first, second, etc. may be used herein to describe various elements, these elements should not be limited by these terms. These terms are only used to distinguish one element from another. Thus, a first element discussed below could be termed a second element without departing from the teachings of the present invention.

Embodiments of the present invention are described herein with reference to schematic illustrations of idealized embodiments of the present invention. As such, variations from the shapes of the illustrations as a result, for example, of manufacturing techniques and/or tolerances, are to be expected.

Provided according to embodiments of the present invention are methods of surface coating pharmaceutical drug delivery devices such as pharmaceutical capsules and tablets with a medication adherence marker. Also provided are the drug delivery devices formed by such methods and pharmaceutical packaging including the same.

### Medication Adherence Monitoring

While the following methods, packaging, and drug delivery devices may be used for any suitable purpose, they are typically used with breath based medication adherence monitoring, wherein a subject ingests a medication adherence marker (MAM), typically with an active pharmaceutical ingredient (API) or a placebo, and then breathes into a device that detects the MAM, thereby confirming that the subject has ingested the MAM, and therefore the API or placebo. As such, according to some embodiments of the invention, provided are methods of medication adherence monitoring wherein a subject ingests a pharmaceutical tablet or capsule coated with a volatile medication adherence marker (MAM) according to an embodiment of the invention, waits a predetermined time period, and then breathes into a medication adherence device that detects the MAM (presence and/or concentration) in the subject's breath. The medication adherence device, or another device in communication therewith, may analyze the presence and/or concentration of the MAM in the subject's breath to confirm that the pharmaceutical tablet or capsule has been ingested by the subject. Additional information regarding such methods is described in the patents and patent applications referenced elsewhere herein.

In the present application, the medication adherence marker (MAM) is a volatile compound that is adsorbed to the surface of a drug delivery device such as a pharmaceutical tablet or capsule and used to provide an indication of whether an individual has ingested the medication (or empty pharmaceutical capsule, e.g., in a double blind clinical trial). In some embodiments of the invention, the MAM is a chemical compound that after dissolution of at least part of the capsule in the individual's stomach will produce a detectable marker in the individual's breath. In some cases, the detectable marker is the MAM itself, but in other cases, it is a metabolite or other compound produced by the MAM. Examples of MAMs, devices used to detect markers, and methods of detection can be described in U.S. Pat. No. 7,820,108, and U.S. Publication Nos. 2014/0294675, 2010/0255598 and 2014/0341983, which are herein incorporated by reference in their entirety. In particular embodiments, the MAM comprises a secondary or tertiary alcohol, such as 2-butanol, which is a considered a generally regarded as safe (GRAS) compound. In another embodiment, the MAM comprises a secondary or tertiary alcohol that is isotopically-enriched, e.g., with one or more deuterium atoms. The term “volatile” refers to a compound that has sufficient vapor pressure at standard temperature and pressure such that MAM vapors from a MAM reservoir (defined below) can adsorb to the surface of a drug delivery device in sealed pharmaceutical package in sufficient amounts that the drug delivery device can be used in medication adherence monitoring.

As described herein, the drug delivery device may be referred to as a pharmaceutical tablet or capsule, but the skilled artisan will understand that any suitable drug delivery device may be used. Any suitable capsule material may be used, including an animal-based material such as gelatin (e.g., a hard shell gelatin material), vegetarian or vegan-based materials (e.g., HPMC). Commercially available capsules may also be used. For example, standard animal-based commercial capsules that may be used include those that are used to encapsulate solids, such as, e.g., Coni-Snap® (Capsugel, Inc.), Quali-G™ and Prism-G™ (Qualicaps, LLC) and those that are used to encapsulate liquids, such as, e.g., LiCaps® (Capsugel, Inc). Standard vegetarian commercial capsules include Vcaps® (Capsugel, Inc.) and other hypromellose-based capsules. Non-standard capsules may also be used. For example, over-encapsulation capsules meant for clinical trials/double blind studies may also be used, such as, e.g., DBcaps® (Capsugel, Inc.). Any suitable tablet and/or tablet formulation may be used, except that if such formulations react with the MAM, additional coating layer(s) may need to be added between the tablet and the MAM. Furthermore, in some embodiments of the invention, the tablets or capsules may be coated with a material that increases adsorption of the MAM to the surface of the tablet or capsule. For example, in some embodiments, the tablets or capsules are coated with maltodextrin. This coating (or an additional coating) may also serve to mask, at least to some extent, any unpleasant taste or odor from the MAM.

### Pharmaceutical Packaging

Pharmaceutical packaging according to some embodiments of the present invention includes a pharmaceutical tablet or capsule; and a volatile medication adherence marker (MAM) contained on or in a MAM reservoir external to the pharmaceutical tablet or capsule. The phrase “external to the pharmaceutical tablet or capsule” means that the MAM reservoir is not part of or attached to the pharmaceutical capsule or tablet.

The MAM reservoir is any material or device that can contain the MAM so that it can vaporize and adsorb onto the pharmaceutical tablet or capsule. In some embodiments, the MAM reservoir is a sorbent material, such as small sorbent pad included within the packaging. In some cases the MAM reservoir may be in physical contact with the pharmaceutical tablet or capsule (i.e., touching). In other embodiments, however, the MAM reservoir may not physical contact the pharmaceutical tablets or capsules. For example, in some embodiments, the MAM reservoir (e.g., a sorbent pad having the MAM therein or thereon) is covered with a layer of material (e.g., a dry sorbent pad) or some other physical barrier. The physical barrier should be sufficiently porous or small, however, so that MAM vapors from the MAM reservoir can reach the surface of the pharmaceutical tablet or capsule in sufficient quantities that the pharmaceutical tablet or capsule may be used in medication adherence monitoring.

Any suitable pharmaceutical packaging may be used provide. However, the packaging should be sufficiently sealed so that the MAM in the package is sufficient to adsorb and maintain the MAM on the surface of the pharmaceutical tablet or capsule so that it can be used in medication adherence monitoring. This will to some extent depend on the shelf life of the packaging, but the packaging should usefully maintain the drug delivery device for pharmaceutically acceptable amounts of time, such as up to 4, 6 or 12 months, and in some cases for longer amounts of time (e.g., 24 months). In some embodiments, the pharmaceutical package is a blister pack and the pharmaceutical tablet or capsule and the MAM reservoir (and optionally the barrier) are placed within a blister cavity of the blister pack.

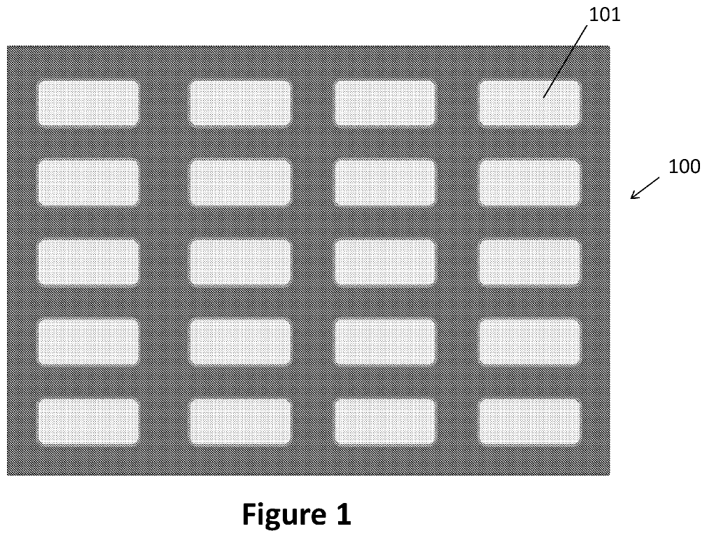

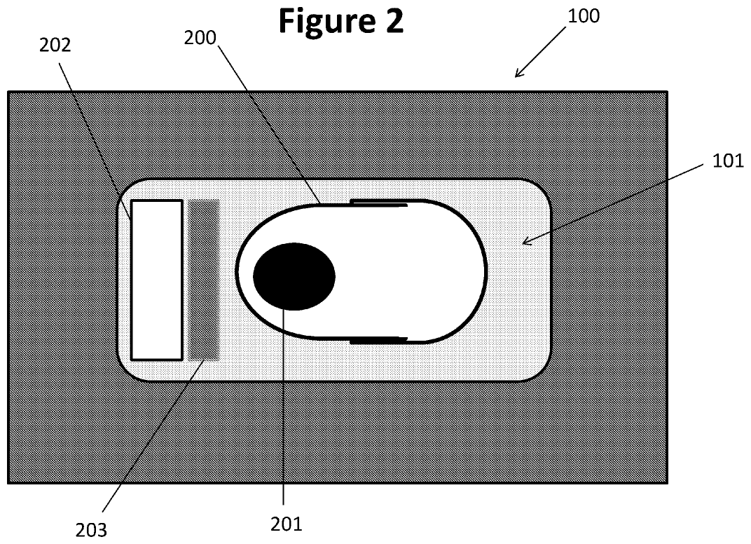

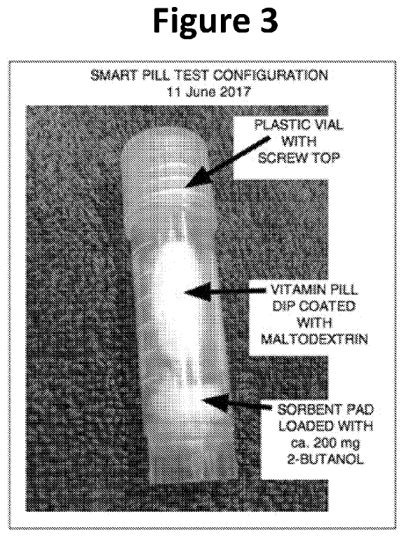

As an example, FIG. 1 shows a typical blister pack 100 having a number of blister cavities 101 defined therein. FIG. 2 is a close up view of one of the blister cavities 101 in the blister pack 100 of FIG. 1. In the embodiment shown in FIG. 2, the blister cavity 101 has a pharmaceutical capsule 200 therein, whereby the pharmaceutical capsule includes active pharmaceutical ingredient (API) 201. The blister cavity 101 also has a MAM reservoir 202, which in this embodiment is a sorbent pad having the volatile MAM absorbed therein or adsorbed thereon. Other types of MAM reservoirs could be used, however. This embodiment also shows a barrier 203, which prevents the MAM reservoir 202 from physically contacting the pharmaceutical capsule 200. For example, the barrier may be a dry sorbent pad. In some embodiments, no barrier is present. The volatile MAM from the MAM reservoir 202 will produce MAM vapor, which will create a vapor pressure in the sealed blister cavity (or other sealed pharmaceutical package), and allow the MAM to adsorb onto the surface of the pharmaceutical capsule 200 (or other pharmaceutical delivery device). As described above, the capsule, tablet or other pharmaceutical delivery device may be pre-treated (e.g., dipped, spray coated, etc.) with a coating that will increase the adsorption of the MAM onto the surface. In particular embodiments, the MAM is 2-butanol and the pharmaceutical tablet or capsule is coated with maltodextrin, which increases the amount of 2-butanol that will adsorb onto the surface of the pharmaceutical delivery device. Some of the MAM in the MAM reservoir 202 may remain in the MAM reservoir 202, while some is adsorbed onto the surface of the pharmaceutical capsule 200. The amount of MAM needed on the surface of the pharmaceutical capsule (or other device) depends on the MAM and the method of detecting such MAM. The amount adsorbed only needs to be sufficient so that when an individual ingests the drug delivery device after opening the package, the MAM (or a metabolite thereof) can be detected in the breath of the individual. Typically the individual will need to ingest the drug delivery device shortly after opening the packaging (e.g., within 5 minutes), but the appropriate amount of time will depend on the specific MAM used.

### Methods of Surface Coating and Drug Delivery Devices

Also provided according to embodiments of the invention are methods of coating pharmaceutical tablets or capsules that include placing the pharmaceutical tablet or capsule in a sealed container with a volatile MAM contained on or in a MAM reservoir external to the pharmaceutical tablet or capsule (or other drug delivery device), and maintaining the seal of the container for a sufficient time to allow MAM vapors from the volatile MAM reservoir to adsorb to onto a surface of the pharmaceutical tablet or capsule. In some embodiments, the sealed container is a pharmaceutical packaging such as a blister cavity in a blister pack, as described above.

As described above, the MAM reservoir may be any suitable storage device for the MAM compound, but in some embodiments, may be a sorbent material having the MAM absorbed therein or adsorbed thereon. The MAM reservoir may or may not be in physical contact with the drug delivery device, and a barrier described above may or may not be present. The drug delivery device may or may not be coated with a coating that increases adsorption of the MAM to the surface of the drug delivery device. Provided herein are the drug delivery devices formed by such methods. Such drug delivery device may be used in combination with any suitable medication adherence monitoring method, but in particular, with the methods described and/or incorporated by reference herein.

The methods described herein may provide a number of benefits for medication adherence monitoring. First, the MAM and the API are physically separated from each other and so the MAM cannot affect the stability or properties of the API. Additionally, the presence of the MAM on the surface of the capsule does not affect the volume or geometry of the API in the tablet or capsule and should have no appreciable effect on the pharmacokinetics of the API. Furthermore, to the patient/individual ingesting the medication, a surface coated tablet or capsule will seem to be a standard capsule or tablet which may improve compliance with the drug regimen. Another benefit is that pharmaceutical companies would not have to modify their capsules or tablets to add the MAM, although they may need or want to coat with a pre-treating solution to increase adsorption of the MAM. However, this may not be needed in some cases, and coating methods such as dipping in maltodextrin solutions may be relatively simple and may not require organic solvents.

### Example 1

Referring to FIG. 3, a vitamin tablet was dipped into a maltodextrin/water solution and the tablet was allowed to dry. A tiny sorbent pad loaded with approximately 200 mg of 2-butanol was added to the bottom of a tiny plastic vial to simulate a blister cavity. The dry coated tablet was then added to the vial and sealed via a screw top. After approximately one week, the vial was opened and the tablet was ingested by an individual. The individual breathed into a miniature gas chromatograph device at regular intervals and a significant quantity of 2-butanone, a metabolite of 2-butanol, was detected in the individual's breath at approximately 10 minutes after ingestion of the tablet. This shows that the 2-butanol from the sorbent pad vaporized in the tiny vial and adsorbed onto the surface of the coated vitamin tablet at sufficient quantities to be detected in the breath of the individual.

In the drawings and specification, there have been disclosed embodiments of the invention and, although specific terms are employed, they are used in a generic and descriptive sense only and not for purposes of limitation, the scope of the invention being set forth in the following claims.

We claim:

1. A pharmaceutical package comprising
a pharmaceutical tablet or capsule; and
a volatile medication adherence marker (MAM) contained on or in a MAM reservoir external to the pharmaceutical tablet or capsule.

2. The pharmaceutical package of claim 1, wherein the MAM reservoir is a sorbent material.

3. The pharmaceutical package of claim 2, wherein the sorbent material comprises the MAM adsorbed thereon or absorbed therein.

4. The pharmaceutical package of claim 1, wherein the package is a blister pack and the pharmaceutical tablet or capsule and the MAM reservoir are contained within a blister cavity of the blister pack.

5. The pharmaceutical package of claim 1, wherein at least some of the volatile MAM is adsorbed onto a surface of the pharmaceutical tablet or capsule.

6. The pharmaceutical package of claim 1, wherein the volatile MAM is an alcohol compound.

7. The pharmaceutical package of claim 1, wherein the volatile MAM is 2-butanol.

8. The pharmaceutical package of claim 1, wherein the MAM reservoir is in physical contact with the pharmaceutical tablet or capsule.

9. The pharmaceutical package of claim 1, wherein the MAM reservoir does not physically contact the pharmaceutical tablet or capsule.

10. The pharmaceutical package of claim 1, wherein the pharmaceutical tablet or capsule has been pre-treated with a coating that increases adsorption of the MAM to the tablet or capsule.

11. The pharmaceutical package of claim 10, wherein coating comprises maltodextrin.

12. A method of surface coating a pharmaceutical tablet or capsule with a volatile medication adherence marker (MAM), comprising
placing the pharmaceutical tablet or capsule in a sealed container with the volatile MAM contained on or in a MAM reservoir external to the pharmaceutical tablet or capsule, and
maintaining the seal of the container for a sufficient time to allow MAM vapors from the volatile MAM reservoir to adsorb to onto a surface of the pharmaceutical tablet or capsule.

13. The method of claim 12, wherein the sealed container is a pharmaceutical packaging.

14. The method of claim 13, wherein the pharmaceutical packaging is a blister cavity of a blister pack.

15. The method of claim 12, wherein the MAM reservoir is a sorbent material having the MAM adsorbed thereon or absorbed therein.

16. The method of claim 15, wherein MAM reservoir is in physical contact with the pharmaceutical tablet or capsule.

17. The method of claim 16, wherein the MAM reservoir is not in physical contact with the pharmaceutical tablet or capsule.

18. The method of claim 17, wherein a barrier is placed between the MAM reservoir and the pharmaceutical tablet or capsule.

19. The method of claim 18, wherein the barrier is sufficiently porous or small to allow for MAM vapors from the MAM reservoir to adsorb onto the surface of the pharmaceutical tablet or capsule.

20. The method of claim 12, further comprising coating the pharmaceutical tablet or capsule with a material that increases adsorption of the MAM onto the surface of the pharmaceutical tablet or capsule.

21. The method of claim 20, wherein the material is maltodextrin.

22. The method of claim 12, wherein the volatile MAM is an alcohol.

23. The method of claim 22, wherein the volatile MAM is 2-butanol.
